(19) United States
(12) Patent Application Publication
(10) Pub. No.: US 2013/0287181 A1
(43) Pub. Date: Oct. 31, 2013

(54) CT APPARATUS AND FRONT COVER OPENING METHOD

(71) Applicants: KABUSHIKI KAISHA TOSHIBA, Tokyo (JP); TOSHIBA MEDICAL SYSTEMS CORPORATION, Otawara-shi (JP)

(72) Inventors: Peng LI, Dalian (CN); Sun Junjie, Dalian (CN); Yu Fengbo, Dalian (CN); Xie Chenghao, Dalian (CN)

(73) Assignees: Toshiba Medical Systems Corporation, Otawara-shi (JP); KABUSHIKI KAISHA TOSHIBA, TOKYO (JP)

(21) Appl. No.: 13/871,496

(22) Filed: Apr. 26, 2013

(30) Foreign Application Priority Data
Apr. 28, 2012 (CN) 201210132991.X

(51) Int. Cl. A61B 6/00

(52) U.S. Cl. CPC A61B 6/44
USPC 378/204

(57) ABSTRACT

According to one embodiment, a CT apparatus includes a gantry main body and a front cover covering a front surface side of the gantry main body, and the front cover includes two cover segments which are held to be configured to open to left and right sides, the cover segments being configured to open or close manually or by being motor-driven by a front cover opening structure.

### CROSS-REFERENCE TO RELATED APPLICATIONS

This application is based upon and claims the benefit of priority from Chinese Patent Application No. 201210132991. X, filed Apr. 28, 2012, the entire contents of which are incorporated herein by reference.

### FIELD

Embodiments described herein relate generally to a CT apparatus and a front cover opening method.

### BACKGROUND

A CT gantry for a CT apparatus includes an openable front cover. It is necessary to safely and conveniently open the front cover of the CT gantry.

With unceasing development and expansion of medical institutions and unceasing updating of large-scale facilities, the structures of CT apparatuses have made great improvements, the performance of detectors has constantly progressed, and the scan velocity has constantly increased, as a result of many years of development. Although there are various types of CT apparatuses, they have basically the same hardware arrangement mainly including three portions, i.e., a scan gantry including an X-ray generation unit and a detector, a control console including a computer, and a movable examination bed. When the operator presses the button, the examination bed slowly moves forward and backward. A display device stands on the upper surface of a box-type operation panel device equipped with a computer which is located near the scan gantry. Operation with the operation panel device will activate the CT apparatus to irradiate/scan a diagnosis region of a patient from a plurality of directions, perform image reconstruction processing based on the projection data obtained from the radiation transmitted through the diagnosis region from the respective directions, and provide a sectional view of the diagnosis region, thereby displaying an image as an examination result on the screen of the display device.

Of all the components of the CT apparatus, the CT scan gantry (to be referred to as the “CT gantry” hereinafter) incorporating precision components such as an X-ray tube, a detector, and the like is the largest and relatively most expensive component. In addition, it is necessary to periodically inspect these components and perform operations such as maintenance. The operator needs to perform these operations after opening the front cover of the CT gantry. In this case, since the front cover of the CT gantry is heavy (20 kg or more) and the rotation fulcrum of the front cover is generally positioned at a level higher than human heights, it is difficult for one operator alone to open the front cover to 90°. When opening the front cover, two operators need to cooperatively perform operation to open the front cover from the vertical position to the horizontal position and support it there. When closing the front cover, the operators need to perform operation in the reverse procedure.

When a front cover 2 of the conventional CT gantry is open, the front cover 2 is held open upward at an angle of 90° with respect to a CT gantry main body 1. In the opening process, as shown in FIG. 12, the two operators lift the front cover 2 connected to the CT gantry main body 1 through a rotating shaft 50 up to the horizontal position when opening the front cover 2, and then insert a support lever b on the front cover 2 into a gantry support portion 52. With this process, the operators open the front cover 2 and fix it at the open position (the position indicated in FIG. 12C).

The conventional front cover opening structure has however the following problems. First, since the front cover of the CT gantry is heavy, two operators are required to cooperatively open the front cover because it is necessary to support the two sides of the cover when opening it. That is, this operation requires much labor. Assume that after opening the front cover, the operator inspects or maintains the interior of the CT gantry. In this case, the operator tends to carelessly raise his/her head and strike his/her head against the lifted front cover 2. That is, this structure is undesirable in terms of safety. In addition, when operating the operation panel after opening the front cover, the operator needs to unfasten the screw of the operation panel on the right side (can unfasten the screw only when maintaining the operation panel on the right side) and to hold the operation panel with one hand and operate the panel with the other hand. This requires much time. In addition, since it is necessary to lift the front cover upward, a wide space is required on the front side (operator side).

In order to solve the above problems, the following has been proposed. That is, patent literature 1 has proposed a CT apparatus including a front cover which covers the front surface of the gantry main body and an opening/closing mechanism which supports the front cover on the gantry main body while allowing the cover to be opened/closed, with opening/closing mechanism using a structure which allows to selectively open the front cover to either of the two sides of the front surface of the gantry main body. This makes it possible to avoid the danger of striking his/her head against the lifted cover. However, since the cover has a large volume and weight, this structure also requires much labor and consumes a large space on the front side (operator side) of the gantry main body.

Patent literature 2 has proposed an apparatus equipped with a mechanism of improving the operability of a front cover which can open on a hinge by using an assist function such as a spring bar. This mechanism makes it necessary to install an expansion mechanism and the like, and hence results in a complicated structure. That is, this mechanism is not suitable for optimization also in terms of labor saving.

### BRIEF DESCRIPTION OF THE DRAWINGS

FIG. 1 is a perspective view of a front cover and gantry main body according to the first embodiment;

FIG. 2 is a schematic plan view of the opening structure of a front cover according to the second embodiment;

FIG. 3 is a perspective view of the rotating means of the front cover;

FIG. 4A is a rear view of a front cover according to the third embodiment, and FIG. 4B is a plan view of the front cover according to the third embodiment;

FIG. 5 is a schematic view showing the opening operation of the front cover according to the third embodiment;

FIG. 6 is a side view of the opening structure of a front cover according to the fourth embodiment;

FIG. 7 is a perspective view showing the opening structure of the front cover according to the fourth embodiment;

FIGS. 8A, 8B, and 8C are side views showing the operation of the opening structure of a front cover according to the fifth embodiment;

FIGS. 9A, 9B, and 9C are side views showing the operation of a ratchet mechanism according to the fifth embodiment;

FIG. 10 is an enlarged perspective view of the ratchet structure according to the fifth embodiment;

FIG. 11 is a perspective view showing a state in which the front cover of a conventional CT gantry is open; and

FIGS. 12A, 12B, and 12C are views for explaining the opening operation of the front cover of the conventional CT gantry.

### DETAILED DESCRIPTION

In general, according to one embodiment, a CT apparatus includes a gantry main body and a front cover covering a front surface side of the gantry main body, and the front cover comprising two cover segments which are held to be configured to open to left and right sides, the cover segments being configured to open or close manually or by being motor-driven by a front cover opening structure.

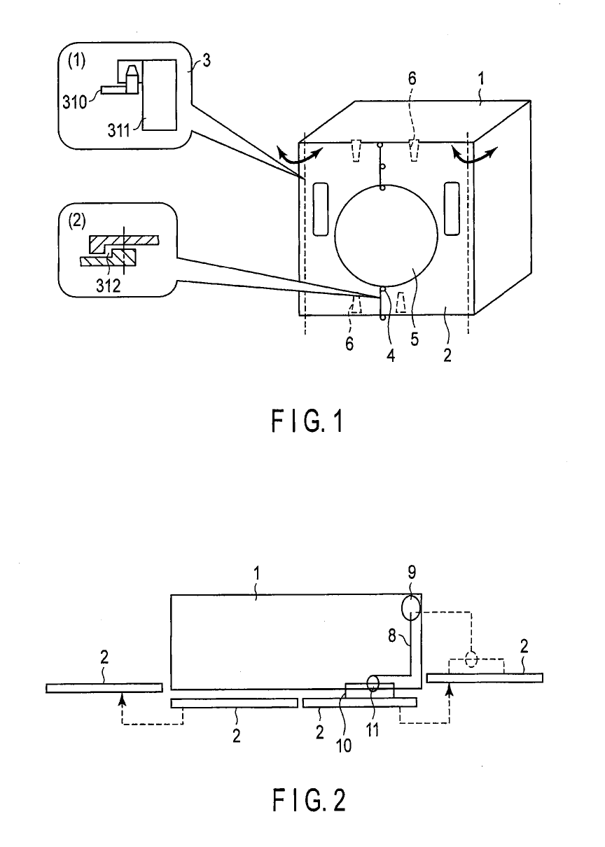

FIG. 1 is a perspective view of a front cover according to the first embodiment. In this embodiment, as shown in FIG. 1, a front cover 2 is constituted by left and right cover segments (two split cover segments) split into two left and right portions. This allows to open the front cover according to the horizontal opening scheme (double-door scheme). In the following description, unless otherwise specified, the “front side” is the side facing the operator of a CT gantry main body 1, the “rear side” is the side opposite to the “front side” of the CT gantry main body 1, the “left side” and “right side” are the left and right sides of the operator who operates facing the CT gantry main body 1, and the “upper side” and “lower side” are the upper and lower sides of the CT gantry main body 1 standing upright on the ground (the upper and lower sides on the drawing).

In the opening structure of the front cover according to this embodiment, one hinge assembly 3 is mounted at each of the upper and lower positions on the two sides of the gantry main body (shown in a partially enlarged view on the upper left portion of FIG. 1). Hinges are connected to the left and right cover segments such that the two hinges on the same side are located on the same axis. This allows the left and right cover segments to rotate about the two axes. As indicated by the enlarged view on the upper left portion of FIG. 1, a conical section of a hinge movable portion 310 of the hinge assembly 3 allows to bear load and facilitates positioning. More specifically, hinge fixing portions 311 are respectively fixed to the left and right cover segments. The hinge movable portions 310 fitted with the hinge fixing portions 311 are fixed to the gantry main body, and the portion of the hinge movable portion 310 which is fitted with the hinge fixing portion 311 is shaped to have a conical section. This allows the hinge movable portion 310 to be easily inserted and fixed in the positioning hole formed in the hinge fixing portion 311, thereby facilitating mounting of the hinge movable portion 310. The gaps between the hinge movable portions 310 and the hinge fixing portions 311 are filled with a lubricant to reduce the rotational frictional force of the split cover segments. When opening the front cover to the left and right, the hinge fixing portions 311 rotate and move together with the front cover relative to the gantry main body.

As indicated by the thick solid lines in FIG. 1, the front cover 2 is constituted by the two cover segments, i.e., the two left and right portions. As indicated by the enlarged view on the lower left portion of the FIG. 1, a crank-shaped structure is formed on the joint line indicated by the thick line between the left and right cover segments to allow the cover segments on the two sides to engage with each other. In addition, the cover segments are fixed with screws 4, and, for example, a rubber gasket (packing) is fitted in a gap 312 between the cover segments (not shown). The gasket can suppress the entrance of dust through the joint line between the cover segments and reduce injuries by the jamming of fingers of servicemen. It is possible to rotate and open the cover segments to the left and right by pulling handgrip portions 5 after removing a mylar sheet 7.

Pins 6 are provided on the two upper sides and the two lower sides near the joint line of the left and right cover segments. Inserting the pins 6 into the corresponding pin holes (not shown) provided in the CT gantry main body 1 will fix the left and right cover segments to the CT gantry main body 1. Fixing the left and right cover segments to the gantry main body in this manner can prevent the left and right cover segments from wobbling relative to the gantry main body. This embodiment uses a total of four pins. However, the number of pins used is not limited to four and may be two or another number.

When opening the front cover 2, first of all, the operator unfastens the screws 4 on the joint line of the left and right cover segments, removes the pins 6 and the mylar sheet 7, and opens the left and right cover segments with handgrip portions 5. The operator closes the front cover following the reverse procedure.

This structure allows the operator by himself/herself to open the front cover. This facilitates maintenance and eliminates the danger of striking his/her head against the front cover. In addition, since the left and right portions of the front cover are coupled tightly, it is possible to prevent the entrance of dust and water. In addition, it is possible to prevent electromagnetic interference to meet EMC demands.

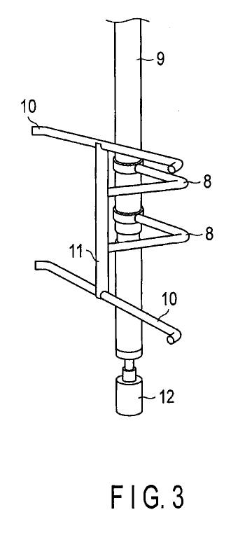

FIG. 2 is a schematic plan view of the opening structure of a front cover according to the second embodiment. FIG. 3 is a perspective view of a rotating means for the front cover according to the second embodiment. The same reference numerals as in the above embodiment denote the same components as those in this embodiment, and a repetitive description of them will be omitted.

The following are the components of the rotating means of the front cover in this embodiment. That is, two support levers 10 are respectively fixed to the left and right cover segments. The two ends of a rotating shaft 11 are rotatably connected to the support levers 10. Two “L”-shaped support levers 8 are disposed in parallel in the vertical direction, with one end of each “L”-shaped support lever 8 being fixed and connected to an intermediate portion of the rotating shaft 11, and the other end being rotatably connected to a fixed shaft 9. However, the number of support levers 8 is not limited to two and may be one or three or more. Alternatively, the two ends of the rotating shaft 11 may be fixed and connected to the support levers 10, and one end of the “L”-shaped support lever 8 may be rotatably connected to an intermediate portion of the rotating shaft 11.

As shown in FIG. 2, the fixed shaft 9 is mounted in the gantry main body 1 and connected to a motor 12 and is rotated and driven.

The opening structure of the front cover 2 according to this embodiment operates in the following manner. That is, driving the motor 12 will make the fixed shaft 9 rotate the overall structure of the rotating means of the front cover and the left and right cover segments. Then, rotating and opening the left and right cover segments to the two sides so as to rotate the support levers 8 about the fixed shaft 9 can open the left and right cover segments to the two sides. Since the operator can open the front cover without biasing it upward, it is possible to save labor, facilitate maintenance, and prevent the operator from striking his/her head against the front cover. At the same time, since the support levers 10 and the rotating shafts 11 rotate in opposite directions, the front cover itself moves while always been kept in a horizontal position. Since it is possible to horizontally move the left and right cover segments to the left and right sides by rotating the support levers 10 about the rotating shafts 11, there is no need to ensure a space on the operator side. This can therefore save labor and a space on the operator side.

Obviously, it is possible to omit the motor in this embodiment and use the structure for manual operation instead of the structure using the motor. At the time of motor-driven operation, the motor rotates the fixed shaft 9. At the time of manual operation, the operator may directly pull the front cover to the two sides.

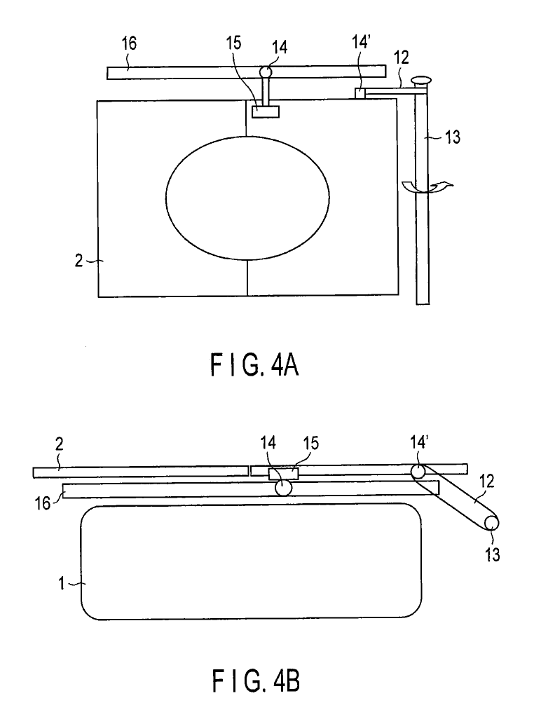

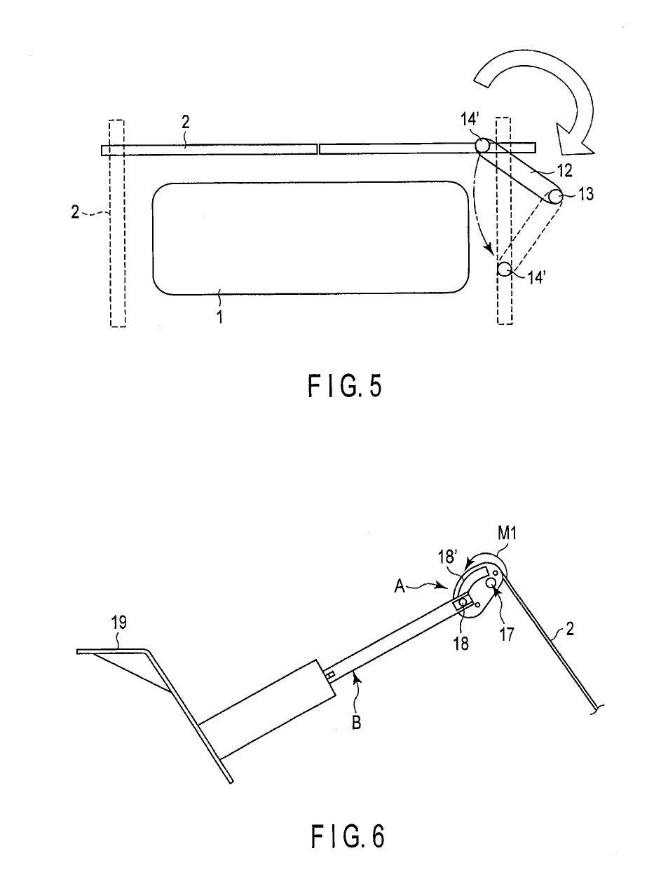

FIG. 4A is a rear view of a front cover according to the third embodiment. FIG. 4B is a plan view of the front cover according to the third embodiment. FIG. 5 is a schematic view showing the opening operation of the front cover according to the third embodiment. The same reference numerals as in the above embodiment denote the same components as those in this embodiment, and a repetitive description of them will be omitted.

In this embodiment, the left and right cover segments are rotatably fixed to fixed shafts 13 installed on the left and right sides through shafts 14′ and fixing devices 12. This allows the left and right cover segments and the fixing devices 12 to rotate relative to the fixed shafts 13. In addition, the left and right cover segments can rotate relative to the fixing devices 12 through the shafts 14′. One end of each of shafts 14 is rotatably fixed to a fixing device 15 mounted on each of the left and right cover segments, and the other end is disposed in a gantry rail groove 16 disposed parallel to the upper end of each of the left and right cover segments of the gantry main body so as to be movable along the gantry rail groove.

The opening structure of the front cover according to this embodiment allows to open the left and right cover segments to the left and right sides by rotating the overall structures of rotating means for the front cover and the left and right cover segments around the fixed shafts 13 by using the fixing devices 12. In addition, since the shafts 14 on the left and right cover segments can only slide along the gantry rail grooves, it restricts the open ends of the left and right cover segments (the intermediate joint line side in FIGS. 14A, 14B, and 15) from approaching the operator side. This reduces the space required on the operator side. The front cover rotates with one end being restricted from moving to the operator side. Furthermore, owing to the position restricting effect of the gantry rail grooves, the shafts 14′ make the front cover 2 and the fixing devices 12 relatively rotate. That is, the left and right cover segments themselves are made to rotate. As a consequence, as shown in FIG. 5, the left and right segments finally move to the positions (each of which is indicated by the broken line in FIG. 5) on the two sides of the gantry main body. This can save labor and spaces on the two sides.

The structure according to this embodiment obtained by dividing the front cover into the left and right cover segments and allows to rotate the left and right cover segments by rotating the overall structure and left and right cover segments by using the fixed shafts 13. The operator needs not open the front cover upward, and hence can save labor and facilitate maintenance. In addition, this structure prevents the operator from striking his/her head against the front cover. At the same time, the operator rotates the left and right cover segments about the fixed shafts 13 by the shafts 14′ and rotates them to the two sides of the gantry main body through 90° while opening the front cover. This structure therefore requires no large space on the operator side. In addition, the position restricting function of the shafts 14 and the gantry rail grooves 16 can move the left and right cover segments to the periphery of the gantry main body without requiring a space on the operator side.

Obviously, this embodiment can use the two schemes, i.e., the motor-driven operation scheme and the manual operation scheme. At the time of motor-driven operation, the motors rotate the fixed shafts 13. At the time of manual operation, the operator directly pulls the front cover to the two sides.

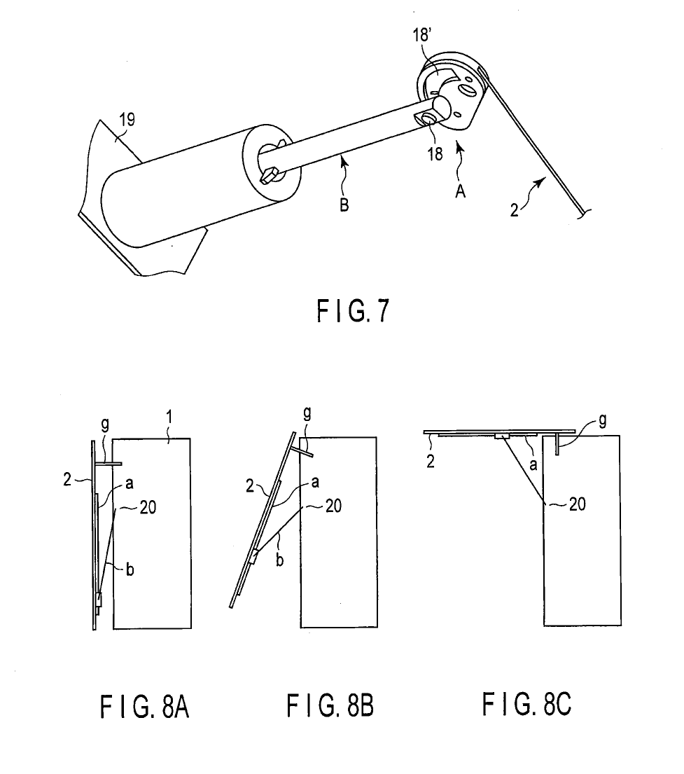

FIG. 6 is a side view of the opening structure of a front cover according to the fourth embodiment. FIG. 7 is a perspective view of the opening structure of the front cover according to the fourth embodiment.

This embodiment additionally includes a spring cam structure near a rotating shaft which connects a front cover 2 to a gantry main body 1 in order to save labor spent to open the front cover.

The spring cam structure includes a cam A fixed and connected to the front cover 2, a front cover rotating shaft 17 around which the cam A fixed to the gantry main body (a position near its upper side) can rotate, a spring B (e.g., a gas spring), and a pin 18 connected to the front end of the spring B, and a sheet metal 19 which fixes the spring B to the gantry main body. The spring B can slide and rotate relative to the cam A by making the pin 18 extend through an arcuated guide groove 18′ formed in the cam A. As described above, the spring B is mounted on the gantry main body 1 by using the sheet metal 19, the pin 18 and the cam A are connected before the spring, and the front cover 2 is fixed to the front cover 2.

When opening the front cover 2, the front cover 2 rotates about the rotating shaft (not shown) mounted on the gantry main body 1 in the counterclockwise direction shown in FIG. 6. At the same time, since the spring B gives a counterclockwise torque M1 (shown in FIG. 6) to the rotating shaft 18 of the front cover through the cam A, it is possible to further save labor when opening the front cover. This makes it possible to easily open the front cover by operation of one operator.

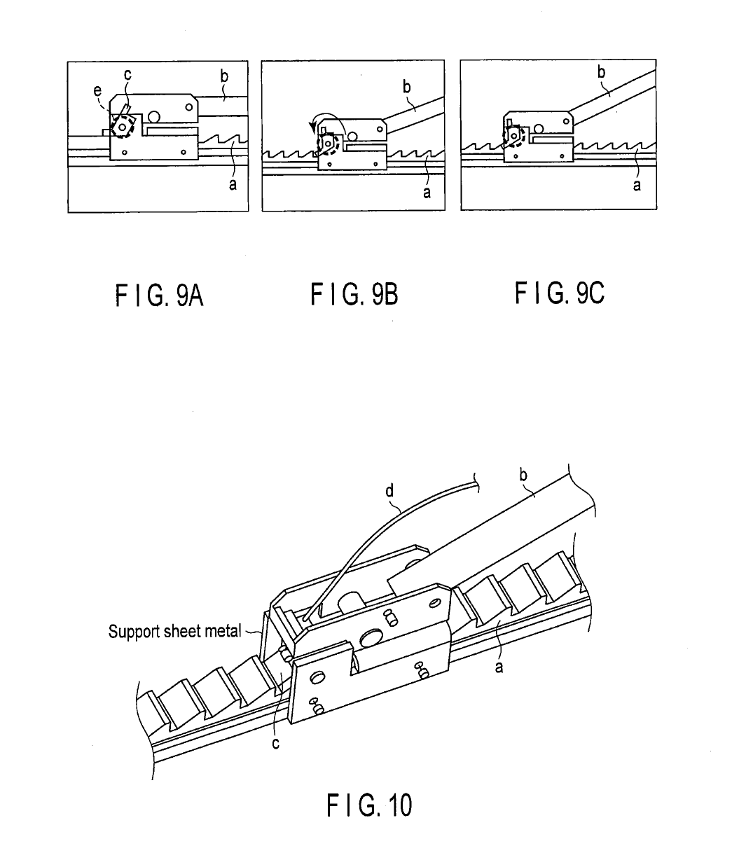

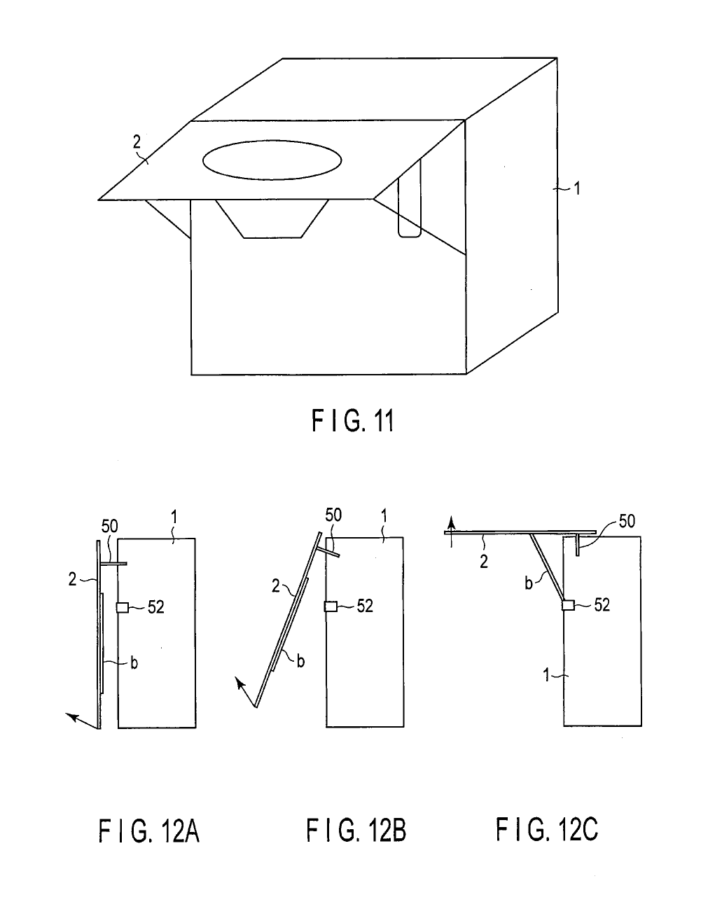

FIGS. 8A, 8B, and 8C are side views showing the operation of the opening structure of a front cover according to the fifth embodiment. FIGS. 9A, 9B, and 9C are side views showing the operation of a ratchet mechanism according to the fifth embodiment. FIG. 10 is an enlarged perspective view of the ratchet structure according to the fifth embodiment. The same reference numerals as in the above embodiment denote the same components as those in the above embodiment, and a repetitive description of them will be omitted.

In this embodiment, based on the above embodiment, a ratchet rack a is fixed to the inside of two sides of a front cover 2, each corresponding support lever b has its rotating shaft 20 fixed to a gantry main body 1, a stopper pin c and a spring e are mounted on the front end of the support lever b (shown in FIGS. 9A, 9B, and 9C), and a control line d is mounted and connected to the stopper pin c (shown in FIG. 10). The stopper pin c is not specifically limited as long as it is manufactured to have a structure which prevents it from slipping off when it slides along the ratchet rack a. In the process of opening the front cover, as described above, when the stopper pin c moves along the ratchet rack a toward a rotating shaft g of the front cover (shown in FIG. 8B), it becomes free from any restriction.

In the process of opening the front cover, the stopper pin c moves upward (to the right side in FIGS. 9A, 9B, and 9C) along the ratchet rack a. Since the angle defined by the stopper pin c and the moving direction is smaller than 90°, the ratchet rack a cannot stop the stopper pin c (shown in FIG. 9B). The stopper pin c is stopped in the process of opening the front cover in the following manner. When the operator releases his/her hand from the front cover, the front cover rotates counterclockwise due to action of gravity. This makes the ratchet rack a move upward (to the right side in FIGS. 9A, 9B, and 9C) relative to the stopper pin c. As a consequence, the gear and the stopper pin c stop (shown in FIG. 9C). In addition, since the stopper pin c is connected to the gantry through the support lever b, when the ratchet rack a and the gantry come to rest relatively, it achieves the stop of the front cover. This makes it possible to open the front cover to an arbitrary angle.

As described above, in the process of opening the front cover, the front cover can be stopped at an arbitrary angle (or a given angle). The presence of a buffering process increases the safety when the operator performs operation alone.

When closing the front cover, the operator can separate the stopper pin c from the ratchet rack a by only turning on the switch (not shown) of the control line d and pulling the stopper pin c with the control line d. This makes it possible to slowly close the front cover owing to the gravity and the effect produced by holding the front cover (shown in FIG. 9A). In addition, turning off the switch of the control line d at an arbitrary angle can stop the front cover at the angle without any burden (shown in FIG. 10).

This structure can stop the front cover at an arbitrary angle, and hence prevents the operator from being injured by the front cover falling down due to the gravity even if the operator carelessly releases his/her hand from the front cover. One operator can therefore perform operation. This increases the operational convenience. Obviously, this structure can be independently installed as the cover segment opening structure of another equipment, and can be applied to any apparatus as long as it is configured to stop the cover segments at arbitrary positions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A CT apparatus comprising a gantry main body and a front cover covering a front surface side of the gantry main body,
the front cover comprising two cover segments which are held to be configured to open to left and right sides, the cover segments being configured to open or close manually or by being motor-driven by a front cover opening structure.

2. The apparatus of claim 1, wherein the front cover opening structure comprises the left and right two cover segments, a plurality of hinge assemblies mounted on the gantry main body and configured to rotate the left and right two cover segments relative to the gantry main body, and a positioning pin which is provided on the cover segment or the gantry main body and engages with a corresponding pin hole provided in the gantry main body or the cover segment.

3. The apparatus of claim 2, wherein joint line portions of the cover segments are formed into structures which engage with each other, a gap between the structures is packed with a gasket, and the joint line portions of the cover segments are fixed to each other with a screw.

4. The apparatus of claim 2, wherein the hinge assembly comprises a hinge fixed portion fixed to the cover segment and a hinge movable portion which fits with the hinge fixed portion and fixed to the gantry main body, and a cross-section of a portion where the hinge movable portion fits with the hinge fixed portion has a conical shape.

5. The apparatus of claim 1, wherein the front cover opening structure comprises a fixed shaft mounted on the gantry main body, a first support lever having an “L” shape, with one end of the support lever being rotatably connected to the fixed shaft, a second support lever having a “U” shape, with two ends of the support lever being respectively fixed to the cover segments, and a rotating shaft having two ends rotatably connected or fixed to the second support lever, with the other end of the first support lever being rotatably connected or fixed to an intermediate portion of the rotating shaft.

6. The apparatus of claim 1, wherein the front cover opening structure comprises a gantry rail groove provided for the gantry main body so as to be parallel to an upper end of the cover segment, fixed shafts provided on two sides of the cover segment of the gantry main body, a fixing device which rotatably fixes each of the cover segments to the fixed shaft, a shaft, with one end on an upper side of the cover segment being rotatably connected to the fixed shaft, and a shaft, with one end being rotatably fixed to the fixing device mounted on the other end of an upper side of the cover segment, and the other end being disposed in the gantry rail groove so as to be movable along the gantry rail groove provided for the gantry main body.

7. A CT apparatus comprising a gantry main body and a front cover covering a front side of the gantry main body,
wherein a spring cam mechanism is provided near a rotating shaft, which connects the front cover to the gantry main body, so as to power-assist opening of the front cover.

8. The apparatus of claim 7, wherein the spring cam mechanism comprises a cam which is fixed and connected to the front cover and in which an arcuated guide groove is formed, a front cover rotating shaft which is fixed to the gantry main body and around which the cam is configured to rotate, a biasing mechanism which is fixed to the gantry main body through a sheet metal and biases in a direction of the front cover when opening the front cover, and a pin connected to a front end of the biasing mechanism, and the biasing mechanism is guided by making the pin extend through the guide groove formed in the cam and slides and rotates relative to the cam along the guide groove.

9. The apparatus of claim 8, wherein the biasing mechanism comprises a gas spring.

10. The apparatus of claim 7, wherein a ratchet mechanism is further provided to stop the front cover at an arbitrary angle in a process of opening the front cover.

11. The apparatus of claim 10, wherein the ratchet mechanism comprises ratchet racks respectively fixed inside two sides of the front cover, support levers respectively corresponding to the ratchet racks, with rotating shafts thereof being fixed to the gantry main body, a stopper pin and a spring which are mounted on a front end of the support lever, and a control line connected to the stopper pin.

12. The apparatus of claim 11, further comprising a switch device configured to control the control line.

13. A front cover opening method for a CT apparatus, wherein a front cover is divided into two cover segments so as be configured to open to left and right sides, and the front cover is configured to be opened in leftward and rightward directions by pulling handgrip portions provided for the cover segments.

14. The method of claim 13, wherein the front cover is configured to be opened so as to be translated to left and right by pulling the handgrip portions provided for the cover segments.

15. The method of claim 13, wherein the front cover is configured to be opened so as to rotate to two sides after translating to left and right by pulling the handgrip portions provided for the cover segments.

16. The apparatus of claim 8, wherein a ratchet mechanism is further provided to stop the front cover at an arbitrary angle in a process of opening the front cover.

17. The apparatus of claim 9, wherein a ratchet mechanism is further provided to stop the front cover at an arbitrary angle in a process of opening the front cover.
